(19) United States
(12) Patent Application Publication
(10) Pub. No.: US 2003/0220630 A1
(43) Pub. Date: Nov. 27, 2003

(54) Methods and systems for treating presbyopia via laser ablation

(76) Inventors: Jui-Teng Lin, Oviedo, FL (US); Colette Cozean, Lake Forest, CA (US); Nick Mamalis, Salt Lake City, UT (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/293,793

(22) Filed: Nov. 12, 2002

Related U.S. Application Data
(63) Continuation-in-part of application No. 09/820,832, filed on Mar. 30, 2001.
(60) Provisional application No. 60/331,497, filed on Nov. 9, 2001.

(51) Int. Cl. A61B 18/20

(52) U.S. Cl. 606/5

(57) ABSTRACT

A method for treating presbyopia by performing laser ablation of the sclera involves making incisions into the sclera which are filled in with material which is more elastic than the original scleral tissue when the incisions heal. Such incisions may be placed radially about the eye in the scleral region outside the limbus of the eye. The increased circumferential diameter and flexibility of the treated portion of the sclera, along with the overall greater volume of the eye, can allow for an increase in the accommodation which can be produced in the eye via the action of the ciliary muscles on the lens and front portion of the sclera.

### RELATED APPLICATIONS

This application claims priority under 35 U.S.C. §119(e) from Provisional Application No. 60/331,497, filed Nov. 9, 2001, the entirety of which is hereby incorporated by reference herein. This application is also a continuation-in-part of copending application Ser. No. 09/820,832, filed Mar. 30, 2001, the entirety of which is hereby incorporated by reference herein.

### BACKGROUND OF THE INVENTION

1. Field of the Invention

The present invention relates in general to systems and techniques for improving the vision of a patient, and more particularly to systems and techniques for treating presbyopia via laser ablation of the scleral tissue of the eye.

2. Description of the Related Art

Presbyopia is a common condition in which the eye loses the ability to focus effectively at nearer distances. Presbyopia becomes more common with increasing age, and is generally considered to be caused by a reduction in the amount of curvature which can be induced in the lens of the eye. Such a reduction in the accommodation of the eye is generally associated with changes that occur in the lens and its associated supporting structures and muscles as a person ages.

Historically, treatment of presbyopia has included techniques such as the use of reading glasses to shift the focal range of the presbyopic eye, surgical methods such as refractive surgery called ‘monovision’, and implantation of inter-ocular lenses. However, the use of glasses can be inconvenient, especially if glasses are already required for distance vision correction. Monovision, in which one eye is treated to provide distance vision while the other is treated to provide close focus, causes a patient to lose depth perception. Furthermore, surgical implantation of multi-focal lenses is still being clinically tested and works in essentially the same manner as multi-focal eyeglasses, i.e., there is no actual increase in the patient's range of accommodation, merely a shifting of the accommodation range to allow focus on closer objects at the expense of focus on farther objects.

Therefore, there is a continued need for improved systems and techniques for improving the ability of presbyopic patients to be able to focus effectively on nearby objects while minimizing the effect on their distance vision.

### SUMMARY OF THE INVENTION

One aspect of the present invention is a surgical method for treating eyes with presbyopia. The method involves increasing the circumferential diameter of the sclera such that the range of accommodation of the eye is increased. This is accomplished by delivering optical energy to the sclera of the eye to create an incision which extends along the sclera from a point at least 0.5 mm outside the limbus of the eye radially away from pupil. The delivery of optical energy produces a region of thermal damage on each side of the extends no more than 500 microns from the incision.

In another aspect of the method described, the surgery method comprises a technique for increasing the flexibility of the sclera in the region in which the ablation is preformed by the delivery of optical energy as described above.

In another aspect of the methods described, the optical energy is generated by an Er:YAG laser. In various modes, this laser energy may be delivered via a focused beam of the laser or via a contact tip. In some modes, the contact tip may have a conical shape Additional aspects of the method involve tracing the region in which the incision is to be made multiple times with the contact tip in a different orientation with respect to the sclera for each pass.

In a further aspect of the method the cross section of the incision produced has a generally rectangular shape. In additional aspects of the method, the width of the incision is between 400 and 600 microns.

In yet another aspect of the method, the increased circumferential diameter of the sclera in the region of the incision is due to the incision filling in with tissue which is more flexible than tissue of the sclera.

In a different aspect of the method described herein, a method is disclosed in which a presbyopic eye is treated by increasing the range of accommodation produced in a lens of the eye by the action of a ciliary muscle. This is done by increasing the circumferential diameter of the sclera by creating new tissue that is more flexible than the remainder of the sclera. At least one incision is formed in the sclera of the eye via laser ablation, the incision having a depth of about 400-500 microns and a width of about 400-600 microns, and this incision being filled in by the new tissue as the incision heals.

An additional aspect of the method is that the action of the ciliary muscle is able to exert a greater amount of tension on the lens due to the increase in the circumferential diameter of the eye. This can allow the lens to flex more in response to the greater tension exerted by the ciliary muscle in a further aspect.

In additional aspects, the action of the ciliary muscle produces a displacement of the lens in the axial direction, due to the increase in the circumferential diameter of the sclera.

Further aspects of the method include the following mechanisms to increase the accommodation of the lens of the eye: an increase in the circumferential diameter of the eye in the region of the incisions, the increased circumferential diameter allowing the lens of the eye to shift forward; an increase in the circumferential diameter of the eye in the region of the incision, the increased circumferential diameter increasing the amount of tension which can be applied by a ciliary muscle of the eye to the lens in order to change the curvature of the surface of the lens; an increase in the circumferential diameter of the eye in the region of the incision, the increased circumferential diameter enlarging the space between the lens and the ciliary muscle, allowing more room for the lens to flex in response to the action of the ciliary muscle; and an increase in the flexibility of the sclera in the region of the incision.

In further aspects, the pressure within the eye pushes the lens forward into the region of increased circumferential diameter, and the action of the ciliary muscle will tend to move the lens forward and backward within the eye.

Further aspects, features and advantages of the present invention will become apparent from the detailed description of the preferred embodiment that follows.

### BRIEF DESCRIPTION OF THE DRAWINGS

The above mentioned and other features of the invention will now be described with reference to the drawings of preferred embodiments illustrating the present vision correction systems and techniques. The Figures are intended to illustrate, but not to limit the invention. The drawings contain the following figures:

FIG. 1 illustrates a cross section of the orb of an exemplary eye.

FIG. 2 illustrates a close up of the lens, ciliary muscles and connecting zonules of an eye in a stretched configuration.

FIG. 3 illustrates the lens of FIG. 2 in a relaxed configuration.

FIG. 4 illustrates a lens which is less convex.

FIG. 5 illustrates a lens which is supported by slack zonules.

FIG. 6 illustrates a cross sectional view of a rectangular scleral incision.

FIG. 7A illustrates a cross sectional view of a “U” shaped scleral incision.

FIG. 7B illustrates a cross sectional view of a “V” shaped scleral incision.

FIG. 8 illustrates a healed rectangular cross section scleral incision.

FIG. 9 illustrates a front view of an exemplary eyeball showing one pattern of scleral incisions which may be used in accordance with one technique described herein.

FIG. 10A illustrates a cross section through the orb of an eye illustrating a lens which has moved forward within the eye.

FIG. 10B illustrates the eye of FIG. 10A with the lens pulled rearward by the action of the ciliary muscle.

FIG. 11A illustrates a flattened lens with the zonules relaxed.

FIG. 11B illustrates the lens of FIG. 11A subject to the normal amount of tension applied by the zonules before correction.

FIG. 11C illustrates the lens of FIG. 11A subject to the increased maximum tension possible after correction.

FIG. 12A illustrates a treated eye with the zonules relaxed.

FIG. 12B illustrates the eye of FIG. 12A showing the increased inward flexing of the sclera due to treatment.

### DETAILED DESCRIPTION OF THE PREFERRED EMBODIMENT

The following description describes various techniques and the associated apparatus of those techniques in accordance with the current invention. Although the embodiments herein are discussed with reference to specific equipment, such as lasers, and in the treatment of a specific condition, it is not intended that the techniques and systems be limited to merely those disclosed uses. Those of skill in the art will recognize that the techniques described are neither limited to any particular type of incision or ablation, nor to the treatment of any single type of ophthalmic condition.

To assist in the description of these systems and techniques, the following directional terms are used. An axis is defined which extends in the anterior-posterior direction through the center of the orb of the eye. This axis extends from the retina at the rear of the eye, through the lens, pupil, and cornea and out through the front of the eye in the direction in which the eye is looking. This direction is referred to as the “axial” or “longitudinal” direction. A “radial” direction is a direction which is generally perpendicular to this longitudinal axis through the eye. The “outward” direction is used herein to refer to a general direction away from the center of the orb of the eye, and more particularly to refer to a direction radially away from the longitudinal axis. “Inward” is used to refer to a direction toward the center of the orb, or toward the longitudinal axis of the eye. “Forward” is used to refer to the direction along the longitudinal axis directed away from the center of the eye toward the pupil, and “rearward” refers generally to the direction along the longitudinal axis directed away from the center of the eye and away from the pupil.

To facilitate a complete understanding of the invention, the remainder of the detailed description describes the invention with reference to the Figures, wherein like elements are referenced with like numerals throughout.

Overview

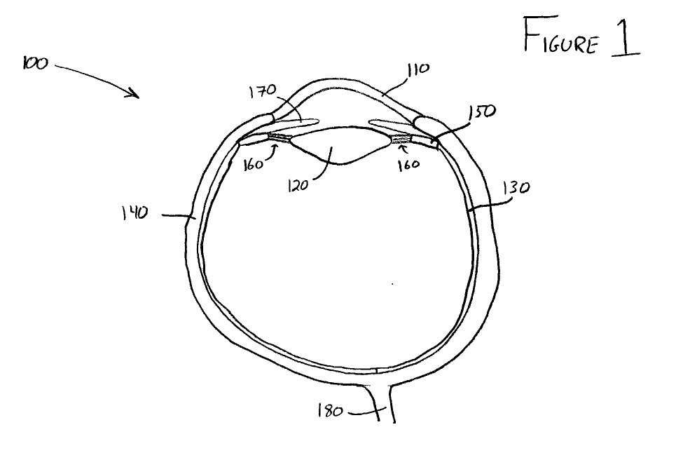

FIG. 1 shows a cross sectional view of an exemplary human eye 100. In the ordinary course of vision, the light reflected from objects passes through the cornea 110, a clear membrane located at the front portion of the eye 100, through the lens 120, and onto the retina 130, located inside the sclerotic layer, or sclera 140. A thin layer known as the choroid is disposed between the sclera and retina, but is not shown in FIG. 1. The lens 120 is slightly flexible, and is capable of being flexed by the action of the ciliary muscles 150 which are connected to the lens by the equatorial zonules 160. The iris 170 is disposed forward of the lens 120. The region of the eye where the optic nerve 180 attaches is located at the rear of the orb of the eye.

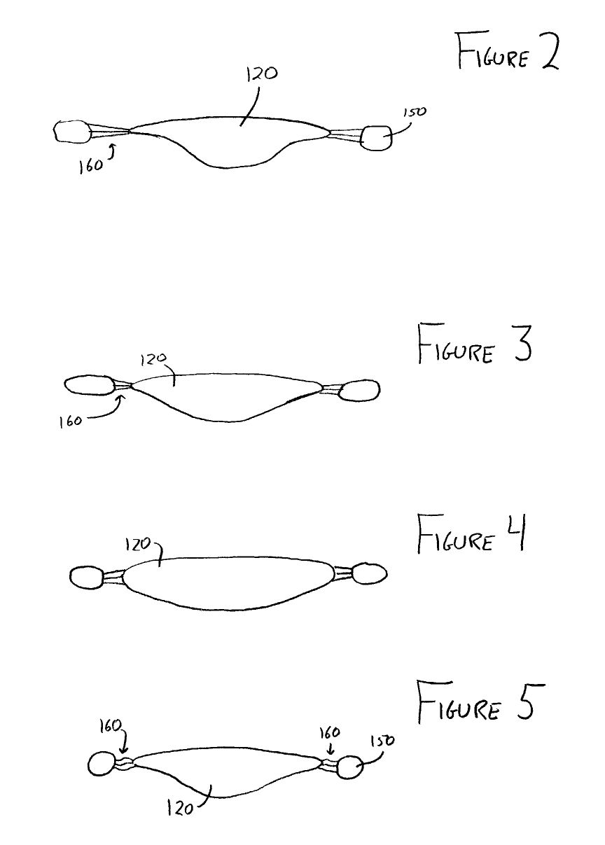

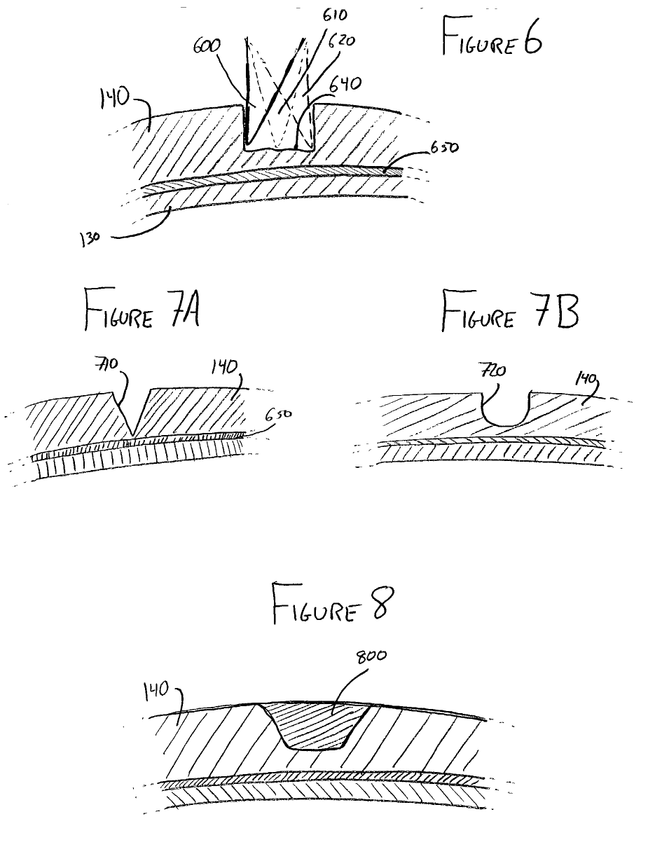

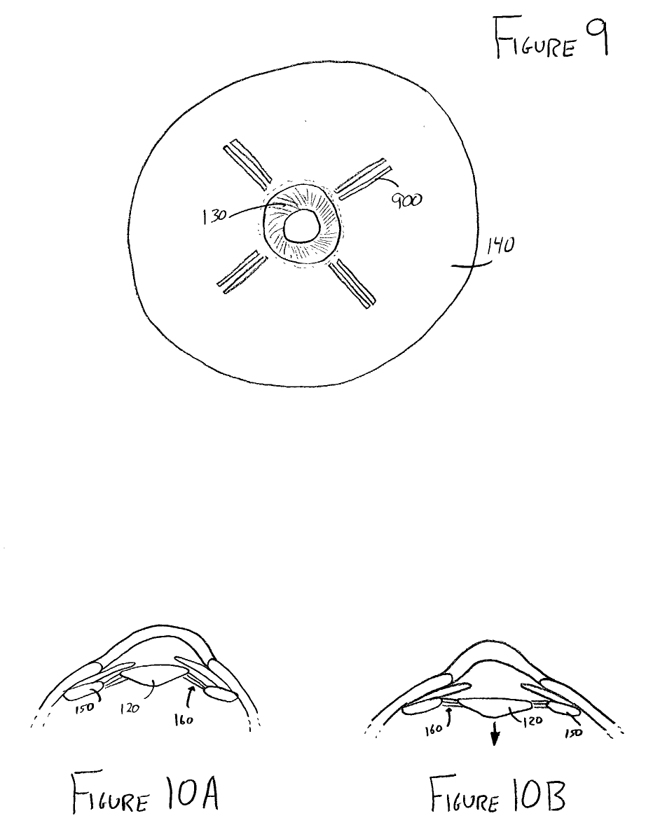

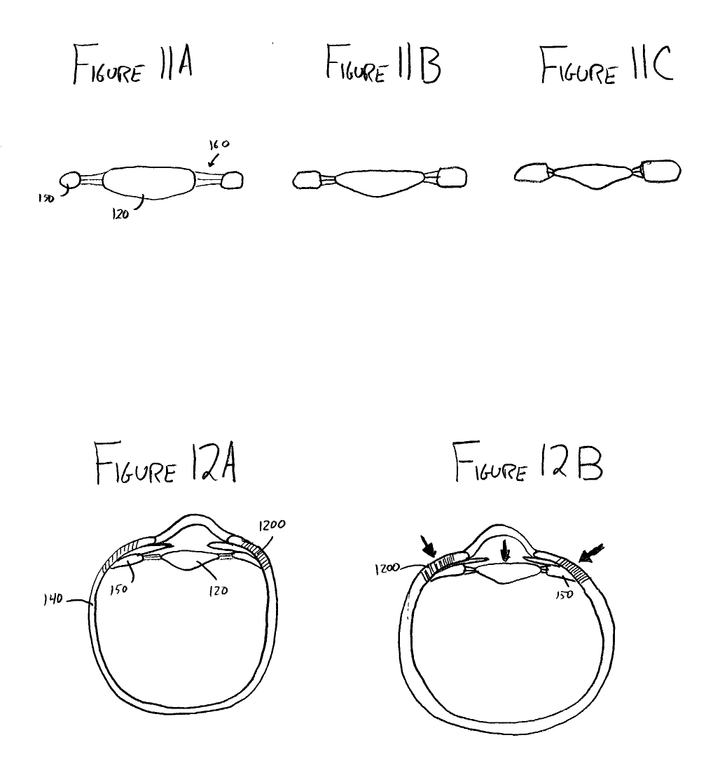

As the ciliary muscles 150 increase or decrease the radial tension upon the lens 120, the lens deforms so as to become more or less convex, changing the focal length of the lens. Increasing the tension in the ciliary muscles 150 tends to stretch the lens radially, flattening the edges of the lens and increasing the curvature near the center of the lens. A stretched configuration of the lens is illustrated in FIG. 2. The ciliary muscle 150 which is attached to the equatorial edge of the lens 120 by the zonules 160 is also shown. As can be seen, the stretching results in a higher optical power of the lens 120, which puts nearby objects into clear focus. By contrast, relaxing the tension in the zonules 160 allows the lens 120 to return to its ordinary shape in which the curvature is more continuous, and the optical power of the lens is less, placing more distant objects into clear focus on the retina 130. Such a relaxed configuration of the lens and associated structures can be seen in FIG. 3.

Through these changes, the light entering the eye can be focused such that objects at a particular distance are clearly focused upon the retina 130. To focus on a different object located a different distance from the eye, the tension applied by the ciliary muscles is altered, and the optical power of the lens 120 is adjusted until the new object is properly in focus. This process of changing the focus of the eye 100 by adjusting the tension of the ciliary muscles 150 to alter the configuration of the lens 120 is known as accommodation.

As mentioned above, presbyopia is a condition where the ability of the eye to accommodate is reduced such that focusing on nearby objects becomes difficult or impossible. The mechanism causing presbyopia is not fully known, but it is generally accepted that with advancing age, the amount of change in optical power of the lens which is produced by the action of the ciliary muscles is reduced. When the amount of change becomes less than that required to produce a high enough optical power to focus on nearby objects, the ability to focus closer than that distance is lost.

This loss of accommodation may be due to one of a variety of reasons. One possibility is that the lens becomes less flexible as it grows, such that the amount of tension required to produce the necessary alteration in the shape of the lens becomes greater than that which can be applied by the ciliary muscles 150. Another possibility is that the lens, as it grows, tends to become thicker and/or less convex along both of its surfaces, reducing the amount of optical power that is produced when the lens is under radial tension by the zonules. Such a flat-surfaced lens is shown in FIG. 4. Still another possibility is that the continued growth of the lens within the area inside the ring of the ciliary muscles results in the lens growing closer to the ciliary bodies surrounding it, thereby introducing some amount of “slack” in the zonules supporting the lens, which reduces their ability to apply tension to the lens. Such slack zonules can be seen in FIG. 5.

Treating presbyopia historically has involved various techniques which shifted the effective accommodation range of the eye from distances for which the available accommodation remained effective, into the range where the accommodation had been lost. For instance, the use of “reading glasses” or bifocals provides an external optical lens which alters the effective optical power of the eye such that nearby objects fall into a range for which there remains sufficient ability to accommodate. However, it is also possible to increase the accommodation of the lens 120 by techniques which are directed to addressing one or more of the above factors which limit the ability of the ciliary muscles to produce the appropriate changes in the optical power or focal length of the lens itself.

For instance, by increasing the elasticity of the sclera 140 of the eye 100, the size of the orb of the eye itself may be increased slightly, producing greater overall internal volume of the eye 100, as well as increasing the radial distance of the ciliary muscles from the axis of the eye. Such a technique can be used to increase the tension which can be produced by the ciliary muscles upon the lens of the eye, as well as increasing the space within which the lens of the eye is free to move. In addition, increasing the size of the sclera 140 of the eye may also result in the ability of lens to move its position along the axis of the eye in response to the action of the ciliary muscles 150, producing an additional mechanism to alter the effective focal length of the lens.

Incisions

In order to produce the suggested changes in the volume or elasticity of the orb of the eye 100 as discussed above, the membranes of the orb of the eye can be incised or ablated. It is possible to create these incisions in a variety of ways, including but not limited to: mechanical incisions, scanning laser systems, contact laser systems, and fiber directed laser systems.

Mechanical incisions may be produced by ordinary sharp implements, such as scalpels. Additional mechanisms of mechanical incision may include devices which simultaneously apply heat or motion to the blade, such as a cauterizing tip or an ultrasonic tip.

Scanning laser systems are those in which a series of mirrors are programmed to direct appropriate bursts of laser energy to specific positions upon the surface of the eye of the patient. Scanning systems may be easily programmed for a variety of different patterns of application of laser energy, and produce highly repeatable results when used appropriately.

Contact laser systems involve the delivery of laser energy through a fiber or tip which is in contact with the area to be treated via the laser. The use of a contact tip allows for precise placement of the laser energy within the tissue to be treated, and also allows for a tightly directed delivery of the energy, which helps minimize the amount of undesired thermal damage to the surrounding tissue.

Fiber beam delivery of laser energy is similar to the contact system described above, but generally provides for a less precise focus of the energy delivered. Such systems may be effective when a broader delivery is desired, such as for use as a cauterizing device.

One way to achieve the desired alteration in the overall volume of the eye, and which may also result in an increase in the elasticity of the sclera 140, is to place incisions radially about the eye in the scleral region outside the limbus. As these incisions heal, they will allow the sclera to expand and assume a greater circumferential radius about the axis of the eye in the area of the incision.

The incisions can also be encouraged to heal such that the incisions are filled in with fibrin and other fibrous tissue which is more elastic than the scleral tissue which it is replacing. Alternate techniques may use incisions which are distributed in patterns other than radially about the axis of the eye. For instance, a “Z” shaped pattern of incisions may also be used to increase the circumferential radius and/or flexibility of the eye.

One particular laser suitable for use in such laser incision/ablation techniques is an Er:YAG laser. The wavelength of this laser causes minimal thermal damage to the surrounding scleral tissue, and can be used to remove a width of about 400-600 microns of tissue in each incision. This laser can be used in conjunction with a conical tip in order to most accurately deliver the laser energy to the appropriate target regions of the sclera for ablation. However, those of skill in the art will recognize that a variety of other lasers and other systems may be used in order to produce effective ablation or incision of the eye. Some other systems which can be used include but are not limited to: excimer lasers, diode lasers, radio-frequency energy delivery systems, electro-cautery systems, and such other systems as would be known to those of skill in the art to ablate tissue while minimizing thermal damage.

It will also be recognized that forms of optical energy other than those produced by a laser maybe used in performing the desired incision and ablation. As used herein, “optical energy” will refer to any form of electromagnetic radiation without regard to whether such electromagnetic radiation would be visible to an eye or not. In this respect, “optical energy” may include x-rays, radio waves, visible light, infrared radiation, ultraviolet radiation, and other forms of energy delivered via photons, whether or not such waves are coherent as in the case of a laser.

In particular, it should be noted that use of the Er:YAG laser with a conical tip to make incisions upon the sclera provides certain benefits which may not be attainable by every other method. One such advantage, as mentioned above, is that the thermal damage caused by the delivery of the laser energy is minimized. While a certain amount of thermal damage may occur directly adjacent to the ablated tissue, the extent of this damage will generally extend less than 500 microns away from the edge of the incision. In most cases, the thermal damage will extend less than 250 microns from the incision. In particular modes of the method described herein, the thermal damage will extend between 5 and 100 microns from the edge of the incision.

One configuration which can produce such beneficial results is to use a laser which delivers between about 10 milli-Joules (mJ) and 50 mJ of energy, more particularly about 20 milli-Joules of energy, in each pulse. The laser may be pulsed at a frequency between 5 and 30 Hz, and more particularly at about 20 Hz.

By minimizing the extent of the thermal damage in this way, there is less of a barrier to regrowth and healing of the scleral tissue. When significant thermal damage is produced, this damaged tissue must be allowed to slough before the incision can properly start healing. Such a delay not only increases recovery time from a procedure, but may also interfere with the type of tissue which fills in the incision during the healing process.

Another advantage of using the Er:YAG laser with a conical tip is that the energy can be delivered very specifically to the desired region of the sclera 140, and the tip may be angled so as to produce a different profile of incision than would be possible with a purely scanning laser system. For example, as shown in FIG. 6, in one application of the Er:YAG laser, the conical tip 600 is applied in multiple passes along the area to be ablated such that the overall cross section 640 of the incision is of a more rectangular form. This is accomplished by angling the tip differently during each pass, the angle being shown in broken lines 610, 620. This can be seen in FIG. 2A. In contrast, incisions 710 created with a single pass of a cutting tip or the use of a scalpel will tend to be “V” shaped as shown in FIG. 7A. Incisions produced via a scanning or fiber delivered laser tend to adopt a “U” shaped configuration 720 as shown in FIG. 7B.

Although both the rectangular 640 and “V” and “U” shaped 710, 720 cross sections may have the same general width and depth, the rectangular cross section has advantages in the manner in which healing takes place. One such advantage is that in a “V” or “U” shaped cut, the width of the cut is narrower at the bottom of the incision. This narrower region will tend to heal much faster than the wider region of the incision, located nearer to the surface of the eye. Because this lower region heals more quickly, it tends not to be filled in with the same sort of flexible, fibrous material that would fill in the more uniform, wider cut of the rectangular cross section incision. In addition, in order to have a sufficient portion of the incision be wide enough to generate the appropriate type of healing, it becomes necessary to cut an incision which is deeper overall than the rectangular cross section cut. This increases the likelihood of incising so deeply that the scleral layer 140 is perforated and the choroid layer 650 beneath the sclera 140 is inadvertently incised. In addition, “V” shaped cuts tend not to expand significantly as they heal, reducing the beneficial effect in enlarging the sclera. Rectangular cuts tend to widen at the top as they heal (see FIG. 8).

In a rectangular incision approximately 400-600 microns in width, the incision will begin healing by first filling in with fibrin and then with other fibrous tissue. Although this tissue will eventually fill in the entire incised area, the resulting healed incision will generally differ from the pre-incision tissue in two ways. First, the healed incision will be slightly wider than the incised area was prior to the tissue ablation. This can be seen in FIG. 8. For example, even though the width of the initial laser incisions may be only about 400-600 microns wide, the healed incision may often be up to 900-1200 microns wide. This is because as the tissue fills in and heals the incision, the reduced overall thickness of the sclera in the incised region will tend to allow the sclera 140 to flex more and expand slightly during the healing process. Second, the fibrous material 800 which fills in the incised region during the healing process will generally be of a greater flexibility than the original scleral tissue which was removed. This combination can result in an overall increase in the elasticity of the region of the sclera surrounding the incision.

Although cuts which are wider or deeper may be used, such variations in the width and depth of the ablated region may result in changes in the nature of the tissue that fills the incision, the time required for the incision to heal, and the overall amount of increased flexibility or circumferential diameter attained after the incisions are fully healed. In particular, such incisions made by laser delivery of optical energy tend to heal in a different manner than cuts of a similar size made by purely mechanical methods, such as scalpels.

Treatment Techniques

The general procedure for performing a scleral ablation in accordance with one embodiment of these techniques is now described. Generally speaking, the procedure can be performed using any appropriate incision device, including but not limited to those lasers and mechanical systems described above. Although it will be understood that a variety of incision mechanisms may be used, the technique described herein will use an Er:YAG laser whose energy is being delivered through a conical contact tip.

First the appropriate anesthesia is applied to eye and, if necessary, the surrounding regions. This may preferably comprise either a peribulbar anesthetic plus a local anesthetic applied to the area of the ciliary body, or a topical anesthetic applied 20-30 minutes prior to surgery. In addition, appropriate topical anesthetic may be applied during the procedure as needed. Suitable anesthetics include but are not limited to: Lidocane with epinephrine/margaine, Tetracane, Astrogel containing Lidocane, and Propericaine. Acuflux, Acular and Amaphcona, as well as other anti-inflammatory or anti-biotic agents may also be applied in order to prevent or minimize complications associated with the procedure, as is understood in the art.

Appropriate protectors and specula may be placed on or about the eye in order to properly hold the eyelids away from the area to be treated, and to prevent any accidental contact with portions of the eye not to be treated. For instance, for scleral ablation, it is generally desirable to place an appropriate corneal protector in position over the cornea of the patient's eye so as to minimize the chance of accidental scratches on the cornea.

The eye is appropriately marked to indicate the regions in which the incisions will be made, and the conjunctiva is cut in these regions to allow access to the scleral layer beneath the conjunctiva. Cauterization can be applied if needed to prevent bleeding while the conjunctiva is open. Then the appropriate scleral ablation may be performed. It is also possible to not cut the conjunctiva prior to performing the ablation and simply to use the laser ablation system to cut the conjunctiva at the same time as the scleral ablation is performed.

Although the exact number and length of incisions to be made may be varied as necessary to fit the needs of the particular patient, it is generally desirable that the incisions be placed in a generally axial-symmetric pattern about center of the eye. In one preferred technique eight incisions may be made in four pairs, with each incision running from about 0.5 mm outside the limbus of the eye (the point where the iris can no longer be seen through the cornea) radially away from the pupil for a length of about 2 to 6 mm. Most commonly, the incisions will be in the range of 4 to 5 millimeters, and in particular embodiments can be about 4.5 mm in length. If ablation is being used, the scleral tissue may desirably be ablated to a depth of about 80% of the overall scleral thickness, generally about 400-500 microns.

One example of such a pattern of incisions 900 is shown in FIG. 9. The incisions 900 are disposed pairwise about the pupil of the eye, separated by approximately 90°. In the illustrated pattern, the incision pairs are disposed roughly 45° from the horizontal and vertical. This helps to avoid any complications associated with the higher density of blood vessels near the nose, and also allows the cuts to be made in portions of the sclera which will tend to be protected by the eyelids after the surgery has been performed. It will be understood, however, that other arrangements may be used as necessary if the benefits would outweigh the particular placement of the incisions 900 illustrated in FIG. 9.

As noted above, the ablation may be performed using a scanning laser mechanism, a bare fiber in a contact or non-contact mode, or a fiber with a conical contact tip. It will also be understood that many lasers which are ordinarily used only for cauterization may be used to perform ablation if the laser energy is delivered in an appropriate manner for ablation (e.g., via a conical tip) rather than in the manner normally used for cautery (e.g., a delivery fiber).

If a conical tip is being used, the ablation region may be passed through more than once with the tip in varying orientations toward the sclera in order to properly form the desired cross section of the ablation region, as discussed above with reference to FIG. 6.

Once the appropriate ablation has been performed, the conjunctiva may be closed and appropriate after-care, such as treatment via Acular and Acuflux, may be performed. It is also generally desirable that the patient proceed to use the eye normally for vision beginning within the first 24 hours after the treatment is performed. This not only allows the sclera to expand more easily as it moves during normal usage of the eye, but also allows the ciliary muscles to begin to strengthen after a period of time in which they may have gone unused.

Effect of Treatment

As discussed above, the general result of the treatment described can be to increase the circumferential diameter, elasticity and flexibility of the sclera in the region in which the ablation was performed. One result of this additional flexibility is that the existing pressure within the orb of the eye will tend to extend this more elastic region of the sclera outward. This will result in a general increase in the overall volume of the interior of the chamber of the eye. In particular, this will result in the radius of the eye becoming larger in the region where the ablation was performed, i.e. in the region of the ciliary bodies.

The method described above for the laser ablation of the sclera does not effect the cornea of the eye, and preferably will not alter the cornea in any way. It should also be noted that although the techniques described herein may be used to correct presbyopia, they may also have slightly different effects on eyes which also suffer from other focal deficiencies. For example, although there is little effect upon myopia by the described treatment, the expansion of the circumferential radius of the sclera by these treatments may also be effective in treating hyperopia. If such a technique is used on a patient effected by both hyperopia and presbyopia, the amount of benefit in the treatment of the hyperopia condition will come at the expense of less correction of the presbyopic condition.

This increase in the radius of the sclera near the ciliary bodies allows the ciliary muscle to adopt a greater overall size and increases the distance between the ciliary bodies and the equatorial portion of the lens 120 to which the ciliary bodies are attached. This increased distance allows the ciliary muscles to produce a greater tension upon the equatorial region of the lens. Such increased tension allows greater deformation of the lens, thereby increasing the range of accommodation which can be achieved via the action of the ciliary bodies upon the lens.

Another effect of the increased radius of the eye in the region of the ablation is to allow the lens of the eye to shift forward within the eye with respect to the retina 130. This forward motion of the lens alters the distance between the lens and the retina, effectively changing the focal length of the eye as a whole. Such a forward shift is made possible by the increased radius provided by the greater flexibility of the forward portion of the sclera. This forward shift may be caused in part by the fluidic pressure within the chamber of the eye pushing against the rear surface of the lens.

Although the lens 120 may shift, it remains connected to the ciliary muscles 150, which may be used to exert varying amounts of tension upon the equatorial region of the lens of the eye. When the lens has shifted forward, this shift will tend to take up any “slack” which may have developed in the zonules attaching the ciliary bodies to the lens. This is shown in FIG. 10A. By increasing the tension exerted upon the edge of the lens by the equatorial zonules 160, the lens 120 is not only stretched in the radial direction, but may also be pulled back toward the retina 130 as shown in FIG. 10B. The arrow indicates the direction in which the tension in the zonules will tend to move the eye. In this way, the action of the ciliary muscles can now produce actual changes in the focal position of the lens within the eye, as well as changes in the shape, and hence optical power, of the lens 120 itself.

An additional mechanism through which accommodation may be increased due to the increased volume of the orb of the eye is through an increase in the maximum tension which can be applied to the lens by the zonules. The maximum tension applied by the zonules is increased because of the increase in the distance between the ciliary muscle and the lens. Raising the tension which can be applied allows the lens 120 to flex more in response to the action of the ciliary muscles. This can increase the available accommodation because the lens has a tendency to grow less convex (flatter-surfaced) over time (as shown in FIG. 4).

Because the lens adopts this less curved shape as it ages (see FIG. 11A), the same amount of tension being applied by the ciliary muscles 150 to the lens will tend to result in less total curvature of the lens as it grows older (See FIG. 11B). Eventually, the amount of tension which can be applied (prior to correction) may not be sufficient to produce the necessary curvature to achieve the optical power needed for close focus. However, if the greater range of motion of the ciliary bodies allows for a higher overall tension to be applied to the lens, then the lens may be made to flex sufficiently to achieve the necessary curvature even though its starting shape is less curved than it once was. (See FIG. 11C.)

An additional means in which the increased flexibility of the front portion of the sclera can result in greater accommodation is in the increased ability of the eye itself to flex in response to the action of the ciliary muscles. The ciliary muscles are arranged such that they not only exert tension forces on the lens via the zonules connecting the ciliary bodies to the lens, but they also effectively exert an inward tension force upon the forward portion of the eye itself. By attempting to stretch the lens radially, the ciliary muscle also tends to pull inwardly on the regions of the sclera to which the muscle itself is attached. Even in circumstances in which the lens is unable to flex sufficiently to achieve a greater degree of accommodation, the sclera itself will tend to bend inward in the region of increased flexibility. Through this motion of the sclera, the sclera will tend to be pulled back toward the original circumferential radius it had prior to the described treatment. By doing so, the position of the lens will tend to move closer to the retina in response to increased tension on the lens and the forward portion of the eye.

FIG. 12A illustrates the initial position of the lens 120 and sclera 140 after treatment. The treated region 1200 of the sclera 140 is indicated by the hash marks. When the ciliary muscle 150 is used to increase the tension in the zonules 160, it will tend to pull inwardly on the treated region 1200 as well as pulling outwardly on the lens 120. This will tend to cause the treated region 1200 to flex somewhat inwardly, toward the position that the treated region 1200 had prior to treatment. This motion is indicated by the arrows in FIG. 12B. The result is that variation between a position closer to the pre-treatment configuration of the sclera 140 (shown in FIG. 12B) and the post-treatment configuration (shown in FIG. 12A) may be controlled by the action of the ciliary muscle 150.

In addition to increasing the ability of the eye to accommodate sufficiently to focus on nearby objects, the described techniques for achieving such increases in accommodation provide additional advantages over those presbyopia corrections previously available. One particular advantage produced is that the ability to focus on nearby objects is achieved by an actual increase in the range of accommodation possible by the eye, rather than merely a shifting of the available range of accommodation. For instance, through the use of reading glasses, it is possible to apply an overall change to the focal power of the eye. This allows the range of accommodation normally used in altering focus between midrange objects and distant objects to be used for fine tuning the focus of the eye between various near objects. However, while wearing reading glasses, the ability to focus on more distant objects is lost, such a focal adjustment being beyond the range of the available accommodation.

To cope with this, bifocal or multifocal lenses may be worn which provide differing degrees of focal power correction in different portions of the eyeglass lens. However, in each case, the ability of the eye itself to accommodate remains unaltered; it is merely the shifting of the available range of accommodation that is accomplished via the use of the corrective lenses. Similarly, techniques which rely upon altering the eye in such a way to produce a permanent change in optical power of a portion of the eye also result in simply shifting the available range of accommodation, rather than actually increasing the range of accommodation possible.

By contrast, the technique described herein makes possible an actual increase in the range of accommodation of the corrected eye as compared to the uncorrected eye. This allows the possibility of allowing for accommodation sufficient to focus on nearby objects, while still allowing the patient to retain his ability to focus on distant objects without the use of corrective lenses for either condition. Such a technique is not possible via the application of any single optical power correction to the eye, whether such a correction is applied via a contact lens, eyeglasses, or a fixed inter-ocular implant.

An additional advantage of the system described herein is that it does not require invasive surgery in the same manner in which an inter-ocular implant would require. This helps simplify the surgery itself, as well as reducing the amount of time for recovery after surgery, and reducing the likelihood of undesirable complications, such as infections or unanticipated tissue damage.

One particular treatment which has been used is known as anterior ciliary sclerectomy, or ACS, which involves a similar technique to that described above, but makes use of incisions created with a scalpel. Although such incisions can produce temporary changes in the sclera of the eye, the incisions tend to heal very quickly and without any significant permanent change in the size or flexibility of the treated eye. By contrast, the changes produced via laser ablation incisions tend not to regress as the incisions heal.

Another technique based off of such mechanical treatments involves implanting an expander within the sclera to attempt to mechanically force the eye to adopt a different size or configuration. Such techniques involve an invasive surgery and all of the associated complications which may result. In addition, the implantation of a foreign body (i.e., the expander) within the tissues of the eye introduces the possibility of foreign body rejection and other adverse reactions in the adjacent tissues.

The various embodiments of scleral ablation techniques described above thus provide a number of ways to provide safe and effective treatment of presbyopia. In addition, the techniques described may be broadly applied for use with a variety of other ophthalmic conditions.

Of course, it is to be understood that not necessarily all such objectives or advantages may be achieved in accordance with any particular embodiment using the systems or techniques described herein. Thus, for example, those skilled in the art will recognize that the techniques may be developed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. For example, variations in the laser wavelength used to ablate the scleral tissue may be combined with techniques using a different number of cuts. Similarly, the various patterns of ablation, as well as other known equivalents for the described features, can be mixed and matched by one of ordinary skill in this art to produce surgical techniques and equipment in accordance with principles of the system described herein.

Although these techniques and systems have been disclosed in the context of certin embodiments and examples, it will be understood by those skilled in the art that these techniques and systems may be extended beyond the specifically disclosed embodiments to other alternative embodiments and/or uses and obvious modifications and equivalentis thereof. Thus, it is intended that the scope of the systems disclosed herein disclosed should not be limited by the particular disclosed embodiments described above, but should be determined only by the scope of the claims that follow.

What is claimed is:

1. An ophthalmic surgery method for treating a presbyopic eye, the method comprising increasing the circumferential diameter of the sclera such that the range of accommodation of the eye is increased, said increasing comprising delivering optical energy to the sclera of the eye to create an incision which extends from at least 0.5 mm outside the limbus of the eye, the incision extending radially along the sclera, said delivering optical energy comprising producing a region of thermal damage on each side of the incision which extends no more than 500 microns from the incision.

2. An ophthalmic surgery method as in claim 1 wherein the method further comprises increasing the flexibility of the sclera by said delivering of optical energy.

3. An ophthalmic surgery method as in claim 1 wherein the optical energy comprises the output of an Er:YAG laser.

4. An ophthalmic surgery method as in claim 1 wherein said delivery of optical energy comprises placing a contact tip of a probe against the surface of the sclera and tracing the path of the incision to be made.

5. An ophthalmic surgery method as in claim 4 wherein the tip of the probe has a conical shape.

6. An ophthalmic surgery method as in claim 4 wherein the method further comprises tracing the path of the incision at least a second time with the probe disposed in a different orientation with respect to the surface of the sclera.

7. An ophthalmic surgery method as in claim 4 wherein a cross section of the incision has a generally rectangular shape.

8. An ophthalmic surgery method as in claim 7 wherein the width of the incision is between 400 and 600 microns.

9. An ophthalmic surgery method as in claim 1 wherein the increased circumferential diameter of the sclera in the region of the incision is due to the incision filling in with tissue which is more flexible than tissue of the sclera.

10. An ophthalmic surgery method for treating a presbyopic eye by increasing the range of accommodation produced in a lens of the eye by the action of a ciliary muscle, the method comprising increasing the circumferential diameter of the sclera by creating new tissue that is more flexible than the remainder of the sclera, said creating comprising forming at least one incision in a sclera of the eye via laser ablation, the incision having a depth of about 400-500 microns and a width of about 400-600 microns, the incision being filled in by said new tissue as the incision heals.

11. An ophthalmic surgery method as in claim 10 wherein the action of the ciliary muscle is able to exert a greater amount of tension on the lens due to the increase in the circumferential diameter of the eye.

12. An ophthalmic surgery method as in claim 11 wherein the lens flexes more in response to the greater tension exerted by the ciliary muscle.

13. An ophthalmic surgery method as in claim 10 wherein the action of the ciliary muscle produces a displacement of the lens in the axial direction, due to the increase in the circumferential diameter of the sclera.

14. An ophthalmic surgery method as in claim 10 wherein a laser used to supply energy for the laser ablation is an Er:YAG laser.

15. An ophthalmic surgery method for treating a presbyopic eye by incising a sclera of the eye through laser ablation in a pattern such that accommodation of a lens of the eye is increased by at least two of the following mechanisms:
an increase in the circumferential diameter of the eye in the region of the incisions, the increased circumferential diameter allowing the lens of the eye to shift forward;
an increase in the circumferential diameter of the eye in the region of the incision, the increased circumferential diameter increasing the amount of tension which can be applied by a ciliary muscle of the eye to the lens in order to change the curvature of the surface of the lens;
an increase in the circumferential diameter of the eye in the region of the incision, the increased circumferential diameter enlarging the space between the lens and the ciliary muscle, allowing more room for the lens to flex in response to the action of the ciliary muscle; and
an increase in the flexibility of the sclera in the region of the incision.

16. An ophthalmic surgery method as in claim 15 wherein the pressure within the eye pushes the lens forward into the region of increased circumferential diameter.

17. An ophthalmic surgery method as in claim 15 wherein the action of the ciliary muscle will tend to move the lens forward and backward within the eye.
